(19) United States
(12) Patent Application Publication
(10) Pub. No.: US 2026/0265183 A1
(43) Pub. Date: Sep. 10, 2026

(54) METHOD FOR PRODUCING UREA, APPARATUS FOR PRODUCING UREA, AND METHOD FOR IMPROVING EXISTING APPARATUS FOR PRODUCING UREA

(71) Applicant: Toyo Engineering Corporation, Chiba-shi, Chiba (JP)

(72) Inventors: Takahiro YANAGAWA, Chiba-shi, Chiba (JP); Yasuhiko KOJIMA, Chiba-shi, Chiba (JP); Keiji SANO, Chiba-shi, Chiba (JP); Shogo KAWATA, Chiba-shi, Chiba (JP)

(73) Assignee: Toyo Engineering Corporation, Chiba-shi, Chiba (JP)

(21) Appl. No.: 19/165,628

(22) PCT Filed: Apr. 10, 2024
(86) PCT No.: PCT/JP2024/014513

§ 371 Date: Sep. 16, 2025

(30) Foreign Application Priority Data
Apr. 12, 2023 (JP) 2023-064707

(51) Int. Cl. C07C 273/04; B01D 19/00; B01D 53/14; B01D 53/18; B01J 19/24

(52) U.S. Cl. CPC C07C 273/04; B01D 19/0073; B01D 53/1418; B01D 53/1425; B01D 53/1475; B01D 53/18; B01J 19/245; B01D 2252/102; B01D 2256/22; B01D 2257/504

(57) ABSTRACT

Provided is a method for producing urea with a relatively high urea synthesis rate and a relatively low energy consumption. The method for producing urea of the present invention includes a carbon dioxide separation step of absorbing and separating a carbon dioxide-containing gas 10 into a lean solution 20, which is an absorbing solution, to obtain a rich solution 11, a rich solution stripping step of stripping this rich solution 11 to obtain a gas 21 containing high-concentration carbon dioxide, a high-pressure absorption step of obtaining a carbamate solution 25 using this gas 21, and a urea synthesis step of using this carbamate solution 25 as part of raw materials.

### FIELD OF THE INVENTION

The present invention relates to a method for producing urea, an apparatus for producing urea, and a method for improving an existing apparatus for producing urea with a relatively high urea synthesis rate and a relatively low energy consumption.

### BACKGROUND OF THE INVENTION

Conventionally, various technologies for absorbing and separating carbon dioxide from a flue gas have been known. For example, one of the technologies is a method for bringing a flue gas into contact with an ammonia solution (absorbing solution) to absorb and separate carbon dioxide contained in the flue gas into the ammonia solution. Further, through this absorption and separation, an absorbing solution containing high-concentration carbon dioxide, ammonia and water (rich solution) is produced.

On the other hand, in a urea synthesis process, usually, carbon dioxide and ammonia are reacted at a high temperature and under a high pressure to obtain a urea synthesis solution. A method in which the above absorbing solution after the absorption of carbon dioxide (rich solution) is utilized as part of the raw materials for urea synthesis in such a urea synthesis process is known. U.S. Pat. No. 9,428,449 describes such a method. Specifically, the above patent literature proposes a method in which an ammonia production plant and a urea production plant are integrated with one another, such that carbon dioxide in a flue gas produced during combustion in the ammonia production plant is absorbed into an ammonia solution (absorbing solution) to form a rich solvent, and the rich solvent is utilized as part of the raw materials for urea synthesis.

For example, in FIG. 1 of the above patent literature, a flue gas stream 5 produced during combustion in an ammonia production plant 1 is transported to a CAP absorber 10. Further, a mixed stream 11 (a mixed solution of an ammonia stream 9 produced in the ammonia production plant 1 and a lean solvent stream 24, which is an absorbing solution containing low-concentration carbon dioxide) is also transported to the CAP absorber 10. Further, in this CAP absorber 10, carbon dioxide in the flue gas stream 5 is absorbed and separated into the solution of the mixed stream 11 (absorbing solution). The solution after the absorption of carbon dioxide (a rich solvent stream 13 containing high-concentration carbon dioxide) is transported to a urea synthesis section 15. Further, an ammonia stream 8 produced in the ammonia production plant 1 and a carbon dioxide stream 7 separated from a syngas produced in the ammonia production plant 1 are also transported to the urea synthesis section 15. Further, these are used as raw materials for urea synthesis.

In FIG. 2 of the above patent literature, a flue gas stream 25 is transported from a steam reformer unit 14 to a low pressure absorber 29. Further, a lean solvent stream 42 is also transported to the low pressure absorber 29. Further, in this low pressure absorber 29, carbon dioxide in the flue gas stream 25 is absorbed and separated into a solution of the lean solvent stream 42 (absorbing solution). A partial stream 31 of the solution of the lean solvent stream 42 after the absorption of carbon dioxide (a semi rich solvent stream 30 containing carbon dioxide in a relatively high concentration) is transported to a urea production plant 38, and another partial stream thereof is transported to a high pressure absorber 33 together with a partial stream 32 of the lean solvent stream 42. Further, a hydrogen/carbon dioxide stream 28 is also transported to the high pressure absorber 33. Further, in this high pressure absorber 33, carbon dioxide in the hydrogen/carbon dioxide stream 28 is absorbed and separated into a mixed solution of the partial stream of the semi rich solvent stream 30 and the lean solvent stream 42 (absorbing solution). A rich solvent stream 37, which is part of the mixed solution after the absorption of carbon dioxide, is transported to the urea production plant 38. An ammonia stream 43 produced in an ammonia production unit 41 is also transported to the urea production plant 38. Further, these are used as raw materials for urea synthesis.

In FIG. 3 of the above patent literature, a regenerator 44 is added between the high pressure absorber 33 and the urea production plant 38 shown in FIG. 2. The rich solvent stream 37, which is part of the mixed solution after the absorption of carbon dioxide in the high pressure absorber 33, is transported to the regenerator 44. In the regenerator 44, carbon dioxide is separated and purified from the solution of the rich solvent stream 37 to obtain high-purity carbon dioxide. This pure carbon dioxide stream 45 is transported to the urea production plant 38. The ammonia stream 43 produced in the ammonia production unit 41 is also transported to the urea production plant 38. Further, these are used as raw materials for urea synthesis.

### SUMMARY OF THE INVENTION

It is generally known that the presence of a large amount of water in urea synthesis results in a significantly reduced urea synthesis rate as water inhibits urea synthesis due to reaction equilibrium (for example, see Ullmann's Encyclopedia of Industrial Chemistry, Fifth, Completely Revised Edition, 1996, Vol. A27, “Urea” p. 333-365). Accordingly, urea synthesis needs to be performed with a somewhat small amount of water in terms of urea synthesis rates. As an index of the amount of water, a molar ratio of water to carbon dioxide, H/C (water/carbon dioxide), is commonly used. Specifically, a desirable H/C in urea synthesis is a low molar ratio of less than 1.5.

On the other hand, when an ammonia solution is used as an absorbing solution for carbon dioxide, even a rich solution after the absorption of carbon dioxide contains a large amount of water. For example, in the rich solvents described in the above patent literature, a concentration of ammonia is 2 to 12 mol/L (about 3 to 12 mass %) and a concentration of carbon dioxide is 1 to 10 mol/L (about 4 to 27 mass %), and a concentration of water is estimated to be about 56 mol/L (about 61 to 93 mass %) from these mass ratios.

Further, in the method described in FIG. 2 of the above patent literature, the only supply sources of carbon dioxide to the urea production plant 38 are rich solutions (the partial stream 31 of the semi rich solvent stream 30 and the rich solvent stream 37). Accordingly, when urea is synthesized under the coexistence of water contained in the rich solutions (about 56 mol/L [about 61 to 93 mass %] in the composition of the rich solutions), the H/C is estimated to be about 4.1, and the urea synthesis rate is estimated to be significantly reduced.

On the other hand, in the method described in FIG. 1 of the above patent literature, not only the carbon dioxide stream 7, but also the rich solvent stream 13 containing about 56 mol/L (about 61 to 93 mass %) of water is used as a supply source of carbon dioxide to the urea synthesis section 15. Accordingly, the H/C in urea synthesis of FIG. 1 is calculated as a molar ratio of water to a total amount of carbon dioxide in the carbon dioxide stream 7 and the rich solvent stream 13, and thus is a higher molar ratio than the H/C in general urea synthesis using the carbon dioxide stream 7 as the only carbon dioxide source. Accordingly, the urea synthesis rate in the method described in FIG. 1 of the above patent literature is estimated to be lower than in the general urea synthesis method.

As explained above, in the methods described respectively in FIGS. 1 and 2 of the above patent literature, the urea synthesis rates are both estimated to be very low. Note that, when industrial production of urea is performed at such low synthesis rates as those in the respective methods, each device in a urea plant needs to be significantly upsized for decomposition and separation of unreacted substances, resulting in an extremely increased energy consumption.

On the other hand, in the method described in FIG. 3 of the above patent literature, the rich solvent containing a large amount of water is not used as-is as a raw material for urea synthesis, but the pure carbon dioxide stream 45 separated and purified from the rich solvent stream 37 in the regenerator 44 is transported to the urea production plant 38. Accordingly, the H/C in urea synthesis of FIG. 3 is estimated to be lower than those in urea synthesis of FIGS. 1 and 2. On the other hand, while the pressure of the carbon dioxide stream 45 is not specified in the above patent literature, it is considered to be about 2 MPaG in usual (for example, see Energy Procedia, 114, “Chilled Ammonia Process Scale-up and Lessons Learned” 2017 p. 5593-5615, particularly, “Regenerator operates at 19.5 bar g” on p. 5615, where 19.5 bar g is about 2 MPaG). A CO₂ compressor is necessary to introduce such a carbon dioxide stream into general urea synthesis facilities operated at a high pressure of about 15 MPaG. Accordingly, the compression power of a CO₂ compressor is increased in the method described in FIG. 3 in which carbon dioxide is sent to the urea production plant 38 only in gas form, as compared to the method described in FIG. 1 in which carbon dioxide is sent to the urea synthesis section 15 in rich solution form and gas form, and the method described in FIG. 2 in which the whole amount of carbon dioxide is sent to the urea production plant 38 in rich solution form.

In other words, an object of the present invention is to provide a method for producing urea, an apparatus for producing urea, and a method for improving an existing apparatus for producing urea with a relatively high urea synthesis rate and a relatively low energy consumption.

As a result of making intensive studies to achieve the above object, the present inventors found out that it is very effective to strip a rich solution to separate a gas containing high-concentration carbon dioxide, use this gas to produce a carbamate solution, and use this carbamate solution as part of the raw materials for urea synthesis, and reached the completion of the present invention.

The present invention is a method for producing urea, including:
a carbon dioxide separation step of bringing a carbon dioxide-containing gas into contact with a lean solution, which is an absorbing solution containing low-concentration carbon dioxide, ammonia and water, to absorb and separate carbon dioxide contained in the gas into the lean solution to obtain a rich solution, which is an absorbing solution containing high-concentration carbon dioxide, ammonia and water;
a rich solution stripping step of stripping at least part of the rich solution obtained in the carbon dioxide separation step to preferentially gasify carbon dioxide to separate a gas containing high-concentration carbon dioxide, and to turn the rich solution into an aqueous solution containing low-concentration carbon dioxide and ammonia;
a high-pressure absorption step of bringing at least part of the gas separated in the rich solution stripping step into contact with ammonia under a high pressure to obtain a carbamate solution; and
a urea synthesis step of obtaining a urea synthesis solution using the carbamate solution obtained in the high-pressure absorption step as part of raw materials.

Further, the present invention is an apparatus for producing urea, including:
a carbon dioxide separation facility (CO₂-AB) for bringing a carbon dioxide-containing gas into contact with a lean solution, which is an absorbing solution containing low-concentration carbon dioxide, ammonia and water, to absorb and separate carbon dioxide contained in the gas into the lean solution to obtain a rich solution, which is an absorbing solution containing high-concentration carbon dioxide, ammonia and water;
a rich solution stripping facility (RST) for stripping at least part of the rich solution obtained in the carbon dioxide separation facility (CO₂-AB) to preferentially gasify carbon dioxide to separate a gas containing high-concentration carbon dioxide, and to turn the rich solution into an aqueous solution containing low-concentration carbon dioxide and ammonia;
a high-pressure absorption facility (HA) for bringing at least part of the gas separated in the rich solution stripping facility (RST) into contact with ammonia under a high pressure to obtain a carbamate solution; and
a urea synthesis facility (R) for obtaining a urea synthesis solution using the carbamate solution obtained in the high-pressure absorption facility (HA) as part of raw materials.

Further, the present invention is
a method for improving an existing apparatus for producing urea, including
adding to the existing apparatus for producing urea, at least
a carbon dioxide separation facility (CO₂-AB)
which is a facility for bringing a carbon dioxide-containing gas into contact with a lean solution, which is an absorbing solution containing low-concentration carbon dioxide, ammonia and water, to absorb and separate carbon dioxide contained in the gas into the lean solution to obtain a rich solution, which is an absorbing solution containing high-concentration carbon dioxide, ammonia and water, and
a rich solution stripping facility (RST) which is a facility for stripping the rich solution, which is an absorbing solution containing high-concentration carbon dioxide, ammonia and water, to preferentially gasify carbon dioxide to separate a gas containing high-concentration carbon dioxide, and to turn the rich solution into an aqueous solution containing low-concentration carbon dioxide and ammonia
to enable a method for producing urea, including:
a carbon dioxide separation step of bringing a carbon dioxide-containing gas into contact with a lean solution, which is an absorbing solution containing low-concentration carbon dioxide, ammonia and water, to absorb and separate carbon dioxide contained in the gas into the lean solution to obtain a rich solution, which is an absorbing solution containing high-concentration carbon dioxide, ammonia and water;
a rich solution stripping step of stripping at least part of the rich solution obtained in the carbon dioxide separation step to preferentially gasify carbon dioxide to separate a gas containing high-concentration carbon dioxide, and to turn the rich solution into an aqueous solution containing low-concentration carbon dioxide and ammonia;
a high-pressure absorption step of bringing at least part of the gas separated in the rich solution stripping step into contact with ammonia under a high pressure to obtain a carbamate solution; and
a urea synthesis step of obtaining a urea synthesis solution using the carbamate solution obtained in the high-pressure absorption step as part of raw materials.

In the method for producing urea of the present invention, the rich solution obtained in the carbon dioxide separation step is not used as-is as a raw material for urea synthesis, but the gas containing high-concentration carbon dioxide is separated from this rich solution in the rich solution stripping step, and further, this gas is used to obtain the carbamate solution in the high-pressure absorption step. Further, this carbamate solution is used as part of the raw materials for urea synthesis.

As a result, in the method for producing urea of the present invention, the H/C in urea synthesis is low, and the urea synthesis rate is high, as compared to the cases where the rich solutions containing a large amount of water are used as-is as raw materials for urea synthesis (FIGS. 1 and 2 of the above patent literature). Further, owing to the high urea synthesis rate, unreacted substances are so reduced that the unreacted substances can be sufficiently decomposed or separated without upsizing each device in a urea plant, making it possible to perform industrial production, and eliminating the necessity of an increased energy consumption.

Further, in the method for producing urea of the present invention, the gas containing high-concentration carbon dioxide is separated from the rich solution, this gas is used to obtain the carbamate solution, and this carbamate solution is used as part of the raw materials for urea synthesis, and thus, even if high-purity carbon dioxide separated and purified from the rich solution is used together as a raw material and a supply source of carbon dioxide for urea synthesis, the high-purity carbon dioxide can be in a relatively small amount. Accordingly, the energy consumption in the method for producing urea of the present invention is low as compared to the case where the high-purity carbon dioxide separated and purified from the rich solution is used as the only supply source of carbon dioxide for urea synthesis (FIG. 3 of the above patent literature). For example, when the amount of high-purity carbon dioxide is relatively small, the compression power of a CO₂ compressor necessary to introduce this into urea synthesis facilities is reduced. Further, when this high-purity carbon dioxide is not used, a CO₂ compressor is unnecessary.

Therefore, the present invention can provide a method for producing urea, an apparatus for producing urea, and a method for improving an existing apparatus for producing urea with a relatively high urea synthesis rate and a relatively low energy consumption.

### BRIEF DESCRIPTION OF THE DRAWINGS

FIG. 1 A process flow diagram showing an embodiment of the method of the present invention.

FIG. 2 A process flow diagram showing another embodiment of the method of the present invention.

### EMBODIMENTS OF THE INVENTION

### <Carbon Dioxide Separation Step>

In the present invention, a carbon dioxide separation step is a step of bringing a carbon dioxide-containing gas into contact with a lean solution, which is an absorbing solution containing low-concentration carbon dioxide, ammonia and water, to absorb and separate carbon dioxide contained in this gas into the lean solution to obtain a rich solution, which is an absorbing solution containing high-concentration carbon dioxide, ammonia and water. Further, a carbon dioxide separation facility (CO₂-AB) is a facility for performing this carbon dioxide separation step.

A type of the carbon dioxide-containing gas used in the carbon dioxide separation step is not particularly limited. For example, a flue gas generated in a combustion process of a facility of a plant, a power station or the like usually contains a large amount of carbon dioxide. In the present invention, such a flue gas is preferably used as the carbon dioxide-containing gas to separate and utilize carbon dioxide in the flue gas as part of the raw materials for urea synthesis in terms of environmental protection. However, the present invention is not limited thereto. Another gas may be used as the carbon dioxide-containing gas as necessary.

In the carbon dioxide separation step, carbon dioxide contained in the above gas is absorbed and separated into a lean solution to obtain a rich solution. The lean solution is an absorbing solution containing low-concentration carbon dioxide, ammonia and water, and is a solution (ammonia-containing aqueous solution) capable of absorbing and separating carbon dioxide contained in a gas at an appropriate temperature and under an appropriate pressure. This lean solution absorbs carbon dioxide to form a rich solution containing an increased concentration of carbon dioxide (an absorbing solution containing high-concentration carbon dioxide, ammonia and water).

The composition of the lean solution used in the carbon dioxide separation step is not particularly limited. It may be any composition as long as it allows carbon dioxide in the gas to be absorbed. For example, as described in the above patent literature, in the lean solution, a concentration of carbon dioxide is usually 0.2 to 4.0 mol/L (about 1 to 13 mass %), a concentration of ammonia is usually 2 to 12 mol/L (about 3 to 15 mass %), and a concentration ratio between ammonia and carbon dioxide is usually 1:1 to 3:1.

A temperature and a pressure in the carbon dioxide separation step are not particularly limited. Any publicly-known temperature and pressure in a method for absorbing carbon dioxide in a gas using an absorbing solution containing ammonia may be applied. For example, as described in the above patent literature, in the carbon dioxide separation step, the temperature is usually 0 to 25° C., and the pressure is usually 0 to 2.5 MPaG.

The composition of the rich solution obtained in the carbon dioxide separation step is not particularly limited. It may be any composition after the absorption of a desired amount of carbon dioxide in the carbon dioxide separation step. For example, as described in the above patent literature, in the rich solution, a concentration of carbon dioxide is usually 1.0 to 10 mol/L (about 4 to 27 mass %), a concentration of ammonia is usually 2 to 12 mol/L (about 3 to 12 mass %), and a concentration ratio between ammonia and carbon dioxide is usually 1:1 to 3:1.

At least part of the rich solution obtained in the carbon dioxide separation step is treated in a rich solution stripping step described later. Another part of this rich solution may be treated in a regeneration step described later.

In the carbon dioxide separation step, a gas after the absorption and separation of carbon dioxide (for example, a clean gas) may be discharged to the outside of the system. Further, a small amount of ammonia contained in this gas may be recovered and regenerated, and reused for the lean solution or as part of the raw materials for urea synthesis.

A type of a facility used as the carbon dioxide separation facility (CO₂-AB) is not particularly limited. For example, a publicly-known facility for absorbing carbon dioxide in a gas using an absorbing solution (for example, an ammonia solution) can be used. An absorber described in the above patent literature may be used.

### <Rich Solution Stripping Step>

In the present invention, the rich solution stripping step is a step of stripping at least part of the rich solution obtained in the carbon dioxide separation step to preferentially gasify carbon dioxide to separate a gas containing high-concentration carbon dioxide, and to turn this rich solution into an aqueous solution containing low-concentration carbon dioxide and ammonia. Further, a rich solution stripping facility (RST) is a facility for performing this rich solution stripping step.

In the rich solution stripping step, stripping means heating the rich solution under an appropriate pressure to an appropriate temperature to preferentially gasify and separate carbon dioxide contained in the rich solution and return the rich solution to the state of an aqueous solution containing a low concentration of carbon dioxide. The gas obtained by this stripping contains a high concentration of carbon dioxide, a relatively low concentration of ammonia and water (water vapor). The boiling point of carbon dioxide is lower than those of ammonia and water, and the boiling point of ammonia is lower than that of water. Accordingly, the above gas is obtained by adjusting conditions in the stripping such as a temperature or the like.

A temperature and a pressure in the rich solution stripping step are not particularly limited. Any publicly-known temperature and pressure in a method for gasifying and separating a component such as carbon dioxide or the like in an absorbing solution (for example, an ammonia solution) may be applied. However, in the rich solution stripping step, the temperature is preferably 100 to 230° C., and more preferably 130 to 210° C., and the pressure is preferably 1 to 16 MPaG, and more preferably 1 to 2.5 MPaG. Particularly, the pressure is preferably approximately the same pressure as that in a high-pressure absorption step described later in terms of relation with a subsequent step.

The composition of the gas obtained in the rich solution stripping step is not particularly limited. It may be any composition which allows a carbamate solution to be obtained by bringing the gas into contact with ammonia under a high pressure in the high-pressure absorption step described later. However, in the gas, a concentration of carbon dioxide is preferably 70 to 100 mass %, and more preferably 80 to 100 mass %, and a concentration of ammonia is, for example, 0 to 25 mass %.

The composition of the aqueous solution obtained in the rich solution stripping step is not particularly limited. This aqueous solution may be used as-is as the lean solution, or a mixed solution of this aqueous solution and part of the rich solution may be used as the lean solution. For example, as described in the above patent literature, in the lean solution, a concentration of carbon dioxide is usually 0.2 to 4.0 mol/L (about 1 to 13 mass %), a concentration of ammonia is usually 2 to 12 mol/L (about 3 to 15 mass %), and a concentration ratio between ammonia and carbon dioxide is usually 1:1 to 3:1. This lean solution is preferably reused in the aforementioned carbon dioxide separation facility (CO₂-AB). Further, it is also preferable to adjust a concentration of this aqueous solution in advance by adding ammonia and/or water thereto to attain optimal composition as the lean solution for separating carbon dioxide.

A type of a facility used as the rich solution stripping facility (RST) is not particularly limited. For example, a publicly-known facility for separating a component in a solution by stripping can be used.

### <High-Pressure Absorption Step>

In the present invention, the high-pressure absorption step is a step of bringing at least part of the gas separated in the rich solution stripping step into contact with ammonia under a high pressure to obtain a carbamate solution. Further, a high-pressure absorption facility (HA) is a facility for performing this high-pressure absorption step.

When ammonia separately supplied is thus brought into contact with the gas containing high-concentration carbon dioxide under a high pressure, carbamate is produced as an intermediate in the urea synthesis reaction. If a solution containing this carbamate (the carbamate solution) is used as part of the raw materials for urea synthesis in a urea synthesis step described later, urea production can be performed at a relatively high urea synthesis rate and a relatively low energy consumption as compared to the methods of the above patent literature.

In the high-pressure absorption step, an amount of ammonia separately supplied is not particularly limited. It may be any amount at such a level as produces carbamate under a high pressure. However, ammonia is preferably supplied in such an amount that a molar ratio of ammonia to carbon dioxide in the carbamate solution, N/C (ammonia/carbon dioxide), is 2.0 to 3.0.

The pressure in the high-pressure absorption step may be any pressure higher than a vapor pressure of the produced carbamate solution. The pressure is preferably 1.0 to 10 MPaG, and more preferably 1.4 to 2.0 MPaG. A temperature in the high-pressure absorption step is not particularly limited. It may be any temperature at which carbamate is produced. However, the temperature is preferably 90 to 180° C., and more preferably 90 to 120° C.

The composition of the carbamate solution obtained in the high-pressure absorption step is not particularly limited, but usually contains carbon dioxide, ammonia and water. In the carbamate solution, a concentration of carbon dioxide is preferably 20 to 50 mass %, and a concentration of ammonia is preferably 20 to 60 mass %. Note that these concentrations of carbon dioxide and ammonia are concentrations for the respective total amounts of carbon dioxide constituting carbamate and unreacted carbon dioxide, and ammonia constituting carbamate and unreacted ammonia.

Further, in this high-pressure absorption step, for example, a separation gas separated in a separation and purification step described later and an aqueous solution separated in a concentration step described later are preferably also used together as part of the raw materials for obtaining the carbamate solution.

A type of a facility used as the high-pressure absorption facility (HA) is not particularly limited. For example, a publicly-known facility for producing a carbamate solution under a high pressure can be used. Further, at least part of absorption heat generated in the high-pressure absorption facility (HA) can also be used for an absorption refrigerating device for creating a low temperature necessary for the carbon dioxide separation facility (CO₂-AB).

### <Urea Synthesis Step>

In the present invention, the urea synthesis step is a step of obtaining a urea synthesis solution using the carbamate solution obtained in the high-pressure absorption step as part of raw materials. Further, a urea synthesis facility (R) is a facility for performing this urea synthesis step.

In the urea synthesis step, at least the components contained in the carbamate solution (carbamate, carbon dioxide and ammonia or the like) are reacted as raw materials, and further, carbon dioxide and/or ammonia separately supplied are also reacted as raw materials as necessary. Further, not only a reactor directly performing such a synthesis reaction, but also a publicly-known condenser (carbamate condenser) or a publicly-known stripper as described, for example, in JP-A H10-182587 or JP-A 2002-145850 is preferably further used together for the urea synthesis step.

In the urea synthesis step, a rich solution containing a large amount of water is not used, and thus, a molar ratio of water to carbon dioxide in urea synthesis, H/C, is low, and a urea synthesis rate is high, as compared to the case where a rich solution is used as-is as a raw material for urea synthesis. Specifically, the H/C is preferably 1.5 or less. Further, a molar ratio of ammonia to carbon dioxide in urea synthesis, N/C (ammonia/carbon dioxide), is preferably 3.0 to 4.0.

A temperature and a pressure for urea synthesis in the urea synthesis step are not particularly limited. Any publicly-known temperature and pressure in a urea synthesis reaction may be applied. However, the temperature and the pressure for urea synthesis are preferably 170 to 200° C., and preferably 13 to 25 MPaG, respectively.

The composition of the urea synthesis solution obtained in the urea synthesis step (a solution to be treated in the decomposition and purification step described later) is not particularly limited, but usually contains urea, unreacted carbon dioxide, unreacted ammonia and water. In the urea synthesis solution, an amount of urea is preferably 40 to 60 mass %, an amount of carbon dioxide is preferably 0 to 20 mass %, an amount of ammonia is preferably 10 to 30 mass %, and an amount of water is preferably 20 to 30 mass %.

A type of a facility used as the urea synthesis facility (R) is not particularly limited. For example, a publicly-known facility for synthesizing urea under a high pressure can be used. Further, a condenser (carbamate condenser) or a stripper as described above is preferably used together.

In the present invention, the urea synthesis solution obtained in the urea synthesis step is preferably subjected to a desired step, such as, for example, the decomposition and purification step or concentration step explained below or the like, to form product urea.

### <Decomposition and Purification Step>

The decomposition and purification step is a step of separating a separation gas containing carbon dioxide and ammonia from the urea synthesis solution obtained in the urea synthesis step to obtain a purified aqueous urea solution after this separation. Further, a decomposition and purification facility (D) is a facility for performing this decomposition and purification step.

Preferably, part of the aqueous solution separated in the concentration step described later is allowed to act as an absorbing solvent on the separation gas separated in this separation and purification step, and thus, they are used together as part of the raw materials for obtaining the carbamate solution in the high-pressure absorption step as described above.

As conditions or the facility in the decomposition and purification step, for example, conditions or a facility in decomposition and purification publicly known as a subsequent step of urea synthesis can be used.

### <Concentration Step>

The concentration step is a step of evaporating water and minute remnants of ammonia and carbon dioxide from the purified aqueous urea solution obtained in the separation and purification step to obtain a concentrated urea solution, recover the evaporated water, ammonia and carbon dioxide and urea entrained in the evaporated gas as an aqueous solution, and treat excess water to obtain clean treated water. Further, a concentration facility (EV) is a facility for performing this concentration step.

As described above, preferably, part of the aqueous solution separated and recovered in this concentration step (recycle solution) is allowed to act as an absorbing solvent on the separation gas separated in the separation and purification step, and thus, they are used together as part of the raw materials for obtaining the carbamate solution in the high-pressure absorption step.

As conditions or the facility in the concentration step, for example, conditions or a facility in concentration publicly known as a subsequent step of urea synthesis can be used.

In the present invention, it is preferable to separate a high-purity carbon dioxide gas from part of the rich solution, for example, in the regeneration step explained below, and also use this together as part of the raw materials for urea synthesis. In this case, the H/C in urea synthesis can be further reduced, and the urea synthesis rate can be further increased.

### <Regeneration Step>

The regeneration step is a step of separating a high-purity carbon dioxide gas from at least part of the rich solution obtained in the carbon dioxide separation step other than the rich solution to be stripped in the rich solution stripping step. Further, a regeneration facility (CO₂-D) is a facility for performing this regeneration step.

In the regeneration step, a carbon dioxide-containing gas is separated from the rich solution, for example, by a publicly-known separation method such as distillation, stripping or the like. Further, when the carbon dioxide-containing gas contains a minute amount of ammonia, ammonia in the gas is removed, for example, by a purification method such as rinsing with water or the like. As a result, the high-purity carbon dioxide gas is obtained. Further, in the urea synthesis step, this high-purity carbon dioxide gas is also used together as part of the raw materials. When the high-purity carbon dioxide gas is also used together as part of the raw materials, troubles of a compressor due to adhesion of a solid compound of carbon dioxide and ammonia generated during gas compression can be prevented. Further, in the present invention, not only the high-purity carbon dioxide gas, but also the carbamate solution obtained from the gas separated from the rich solution is used as a supply source of carbon dioxide to the urea synthesis step. Accordingly, the amount of the high-purity carbon dioxide can be smaller, and thus, the compression power of a CO₂ compressor is reduced as compared to the conventional method in which a high-purity carbon dioxide gas is used as the only supply source of carbon dioxide.

As conditions or the facility in the regeneration step, for example, publicly-known purification conditions or facility for separating a high-purity carbon dioxide gas from a rich solution can be used. The regenerator described in the above patent literature may be used. Further, part of heat recovered from the inside of a urea plant can also be used as a heat source of the regeneration step.

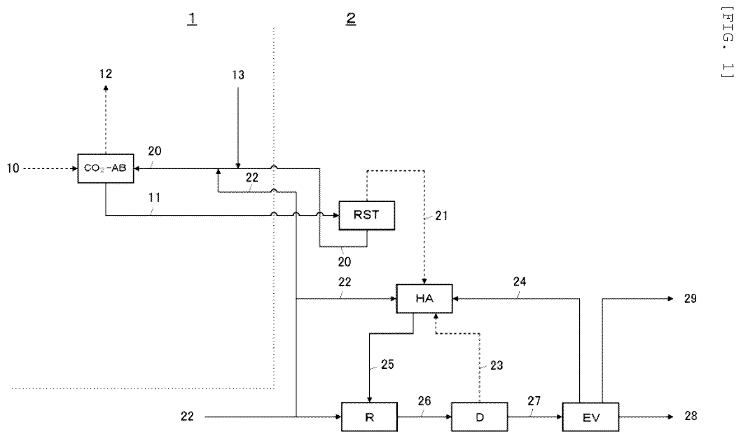

Hereinafter, embodiments of the present invention are explained using drawings. FIG. 1 is a process flow diagram showing an embodiment of the method of the present invention.

An apparatus shown in FIG. 1 includes a carbon dioxide separation facility 1 and a urea plant 2. Further, the carbon dioxide separation facility 1 includes a carbon dioxide absorber (CO₂ absorber) as the carbon dioxide separation facility (CO₂-AB). The urea plant 2 includes a rich solution stripper as the rich solution stripping facility (RST), a high pressure absorber as the high-pressure absorption facility (HA), a urea synthesis facility (R) [which includes at least a reactor and may include a condenser and a stripper], a decomposer as the decomposition and purification facility (D), and an evaporator as the concentration facility (EV).

In FIG. 1, a flue gas 10 is supplied to the carbon dioxide absorber [carbon dioxide separation facility (CO₂-AB)]. A lean solution 20 from the rich solution stripper [rich solution stripping facility (RST)] is also supplied thereto. Further, in the carbon dioxide absorber [carbon dioxide separation facility (CO₂-AB)], the flue gas 10 is brought into contact with the lean solution 20 to produce a rich solution 11 into which carbon dioxide is absorbed and a clean gas 12 from which carbon dioxide is removed. The produced rich solution 11 is supplied to the rich solution stripper [rich solution stripping facility (RST)]. On the other hand, the produced clean gas 12 is discharged to the outside of the system, or subjected to a further scrubbing step and discharged to the outside of the system.

In the rich solution stripper [rich solution stripping facility (RST)] shown in FIG. 1, the rich solution 11 is stripped to produce the lean solution 20 and a gas 21. The lean solution 20 is returned to the carbon dioxide absorber [carbon dioxide separation facility (CO₂-AB)] and reused. Before returning, ammonia 22 and water 13 are each added to this lean solution 20 to adjust in advance a concentration of the lean solution 20.

In the high pressure absorber [high-pressure absorption facility (HA)] shown in FIG. 1, the gas 21, ammonia 22, a separation gas 23 containing carbon dioxide, ammonia and water separated in the decomposition and purification facility D, and a recycle solution 24 from the concentration facility EV are supplied. Further, a carbamate solution 25 is produced under a high pressure. The produced carbamate solution 25 is supplied to the reactor [urea synthesis facility (R)].

In the reactor [urea synthesis facility (R)] shown in FIG. 1, the carbamate solution 25 and ammonia 22 are supplied. These are used as raw materials to synthesize urea and produce a urea synthesis solution 26. This urea synthesis solution 26 is supplied to the decomposer [decomposition and purification facility (D)].

In the decomposer [decomposition and purification facility (D)] shown in FIG. 1, the separation gas 23 containing ammonia, carbon dioxide and water is separated from the urea synthesis solution 26 to form an aqueous urea solution 27. This aqueous urea solution 27 is supplied to the evaporator [concentration facility (EV)], and the separation gas 23 is supplied to the high pressure absorber [high-pressure absorption facility (HA)].

In the evaporator [concentration facility (EV)] shown in FIG. 1, the aqueous urea solution 27 is concentrated to produce a urea product 28 and treated water 29. These urea product 28 and treated water 29 are supplied to subsequent steps.

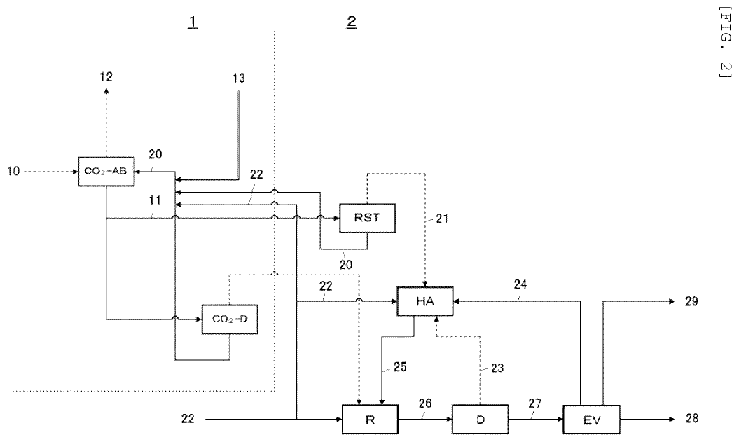

FIG. 2 is a process flow diagram showing another embodiment of the method of the present invention. In an apparatus shown in FIG. 2, a regenerator (CO₂ desorber) as the regeneration facility (CO₂-D) is added to the carbon dioxide separation facility 1 of the apparatus shown in FIG. 1. A CO₂ gas obtained from the regenerator is compressed, and thereafter sent to the urea synthesis facility (R).

### <Method for Improving Apparatus for Producing Urea>

In the present invention, an existing apparatus for producing urea may be improved by adding thereto at least the rich solution stripping facility (RST) [and as necessary the carbon dioxide separation facility (CO₂-AB)] to enable the method of the present invention, in other words, a method for producing urea including, the carbon dioxide separation step, rich solution stripping step, high-pressure absorption step and urea synthesis step explained above.

An embodiment of this improving method is, for example, an improving method including, adding the rich solution stripping facility (RST), and adding or enhancing the high-pressure absorption facility (HA) in the apparatus described in FIG. 1 of the above patent literature (an apparatus with an integrated ammonia production plant and urea production plant). Further, another example is an improving method including, adding to a usual urea production plant the carbon dioxide separation facility (CO₂-AB) and the rich solution stripping facility (RST) [and the high-pressure absorption facility (HA) when the high-pressure absorption facility (HA) is not included therein]. Further, if the regeneration facility (CO₂-D) is further added, a high-purity carbon dioxide gas can also be used together as part of the raw materials in the urea synthesis step.

### EXAMPLES

Hereinafter, the present invention is further specifically explained by examples. However, the present invention is not limited to the examples.

### Example 1

A process simulation in the case of performing urea synthesis in line with the embodiment of the present invention shown in FIG. 1 was performed. The following conditions were used for the process simulation.

### (1) Carbon Dioxide Separation Facility (CO₂-AB)

Composition of lean solution: carbon dioxide 8 mass %, ammonia 7 mass %, water 85 mass %
Temperature: 21° C.
Pressure: 0.01 MPaG
Composition of rich solution: carbon dioxide 11 mass %, ammonia 7 mass %, water 82 mass %

### (2) Rich Solution Stripping Facility (RST)

Temperature: 162° C.
Pressure: 1.7 MPaG
Composition of gas: carbon dioxide 88 mass %, ammonia 4 mass %, water 8 mass %

### (3) High-Pressure Absorption Facility (HA)

Temperature: 107° C.
Pressure: 1.7 MPaG
Composition of carbamate solution: carbon dioxide 38 mass %, ammonia 42 mass %, water 20 mass %

### (4) Urea Synthesis Facility (R)

Temperature: 188° C.
Pressure: 15 MPaG
As a result of performing the simulation under the above conditions, the H/C in urea synthesis was 1.1, and the urea synthesis rate was 58′.

### Example 2

A process simulation in the case of performing urea synthesis in line with the embodiment of the present invention shown in FIG. 2 was performed.

### (1) Carbon Dioxide Separation Facility (CO₂-AB)

Composition of lean solution: carbon dioxide 8 mass %, ammonia 7 mass %, water 85 masse
Temperature: 21° C.
Pressure: 0.01 MPaG
Composition of rich solution: carbon dioxide 11 mass %, ammonia 7 mass %, water 82 mass %

### (2) Rich Solution Stripping Facility (RST)

Temperature: 162° C.
Pressure: 1.7 MPaG
Composition of gas: carbon dioxide 88 mass %, ammonia 4 mass %, water 8 mass %

### (3) High-Pressure Absorption Facility (HA)

Temperature: 107° C.
Pressure: 1.6 MPaG
Composition of carbamate solution: carbon dioxide 42 mass %, ammonia 37 mass %, water 21 mass %

### (4) Urea Synthesis Facility (R)

Temperature: 182° C.
Pressure: 15 MPaG
(5) Regeneration facility (CO₂-D)
Temperature: 146° C.
Pressure: 2.4 MPaG
Suction pressure of CO₂ compressor: 2.5 MPaG
Power of CO₂ compressor: 29 kWh/t-urea

As a result of performing the simulation under the above conditions, the H/C in urea synthesis was 0.6, and the urea synthesis rate was 64%.

### Comparative Example 1

A process simulation in the case of performing urea synthesis using a rich solution as the only supply source of carbon dioxide like the method described in FIG. 2 of the aforementioned patent literature was performed. The following conditions were used for the process simulation.
Composition of rich solution: carbon dioxide 11 mass %, ammonia 7 mass %, water 82 mass %
Composition of carbamate solution: carbon dioxide 13 mass %, ammonia 14 mass %, water 73 mass %
Urea synthesis temperature: 188° C.
Urea synthesis pressure: 15 MPaG

As a result of performing the simulation under the above conditions, the H/C in urea synthesis was estimated to be 4.1.

The H/C values obtained by the simulations of the above examples 1 and 2 and comparative example 1 are shown in Table 1 below. Further, generally estimated values of a suction pressure of a CO₂ compressor and power of the CO₂ compressor of a publicly-known example (general urea plant) are also shown in Table 1 for reference.

TABLE 1

| | | | | Publicly- |
|---|---|---|---|---|
| | | | Comparative | known |
| | Example 1 | Example 2 | example 1 | example |
| Suction pressure of | — | 2.5 | — | 0.08 |
| CO₂ compressor | | | | |
| (MPaG) | | | | |
| Power of CO₂ | 0 | 29 | 0 | 84 |
| compressor | | | | |
| (kWh/t-urea) | | | | |
| H/C in urea synthesis | 1.1 | 0.6 | 4.1 | 0.6 |

As shown in Table 1, the H/C values in examples 1 and 2 are low. On the other hand, in comparative example 1, the H/C is high, and the urea synthesis rate is estimated to be low. Further, the electric power (power of CO₂ compressor) required per ton of urea product in example 2 is less than in the publicly-known example (an example in which a high-purify carbon dioxide gas is used as the only supply source of carbon dioxide) as a required supply amount of a high-purity carbon dioxide gas is less than in the publicly-known example.

### INDUSTRIAL APPLICABILITY

The present invention is useful as a method for producing urea, an apparatus for producing urea, and a method for improving an existing apparatus for producing urea with a relatively high urea synthesis rate and a relatively low energy consumption.

### REFERENCE SIGNS LIST

CO₂-AB carbon dioxide separation facility
RST rich solution stripping facility
HA high-pressure absorption facility
R urea synthesis facility
D decomposition and purification facility
EV concentration facility
CO₂-D regeneration facility
1 carbon dioxide separation facility
2 urea plant
10 flue gas
11 rich solution
12 clean gas
13 water
20 lean solution
21 gas
22 ammonia
23 separation gas
24 recycle solution
25 carbamate solution
26 urea synthesis solution
27 aqueous urea solution
28 urea product
29 treated water

1. A method for producing urea, the method comprising:
a carbon dioxide separation step of bringing a carbon dioxide-containing gas into contact with a lean solution, which is an absorbing solution containing low-concentration carbon dioxide, ammonia and water, to absorb and separate carbon dioxide contained in the gas into the lean solution to obtain a rich solution, which is an absorbing solution containing high-concentration carbon dioxide, ammonia and water;
a rich solution stripping step of stripping, at a temperature of 100 to 230° C. and a pressure of 1 to 16 MPaG, at least part of the rich solution obtained in the carbon dioxide separation step to gasify carbon dioxide to separate a gas containing high-concentration carbon dioxide, and to turn the rich solution into an aqueous solution containing low-concentration carbon dioxide and ammonia;
a high-pressure absorption step of bringing at least part of the gas separated in the rich solution stripping step into contact with ammonia at a temperature of 90 to 180° C. and a pressure of 1.0 to 10 MPaG to obtain a carbamate solution; and
a urea synthesis step of obtaining, at a temperature of 170 to 200° C. and a pressure of 13 to 25 MPaG, a urea synthesis solution using the carbamate solution obtained in the high-pressure absorption step as part of raw materials.

2. The method according to claim 1, further comprising:
a regeneration step of separating a high-purity carbon dioxide gas from at least part of the rich solution obtained in the carbon dioxide separation step other than the part of the rich solution to be stripped in the rich solution stripping step,
wherein in the urea synthesis step, the high-purity carbon dioxide gas is also used together as part of the raw materials to obtain the urea synthesis solution.

3. The method according to claim 1, further comprising:
a separation and purification step of separating a separation gas containing carbon dioxide and ammonia from the urea synthesis solution obtained in the urea synthesis step to obtain a purified aqueous urea solution after the separation; and
a concentration step of evaporating water and minute remnants of ammonia and carbon dioxide from the purified aqueous urea solution to obtain a concentrated urea solution, to recover the evaporated water, ammonia and carbon dioxide and urea entrained in the evaporated gas as an aqueous solution, and to treat excess water to obtain clean treated water,
wherein in the high-pressure absorption step, part of the aqueous solution separated in the concentration step is allowed to act as an absorbing solvent on the separation gas separated in the separation and purification step, such that the obtained carbamate solution is used as part of the raw materials for urea synthesis.

4. An apparatus for producing urea, the apparatus comprising:
a carbon dioxide separation facility for bringing a carbon dioxide-containing gas into contact with a lean solution, which is an absorbing solution containing low-concentration carbon dioxide, ammonia and water, to absorb and separate carbon dioxide contained in the gas into the lean solution to obtain a rich solution, which is an absorbing solution containing high-concentration carbon dioxide, ammonia and water;
a rich solution stripping facility for stripping, at a temperature of 100 to 230° C. and a pressure of 1 to 16 MPaG, at least part of the rich solution obtained in the carbon dioxide separation facility to gasify carbon dioxide to separate a gas containing high-concentration carbon dioxide, and to turn the rich solution into an aqueous solution containing low-concentration carbon dioxide and ammonia;
a high-pressure absorption facility for bringing at least part of the gas separated in the rich solution stripping facility into contact with ammonia at a temperature of 90 to 180° C. and a pressure of 1.0 to 10 MPaG to obtain a carbamate solution; and
a urea synthesis facility for obtaining, at a temperature of 170 to 200° C. and a pressure of 13 to 25 MPaG, a urea synthesis solution using the carbamate solution obtained in the high-pressure absorption facility as part of raw materials.

5. The apparatus according to claim 4, further comprising:
a regeneration facility for separating a high-purity carbon dioxide gas from at least part of the rich solution obtained in the carbon dioxide separation facility other than the part of the rich solution to be stripped in the rich solution stripping facility,
wherein in the urea synthesis facility, the high-purity carbon dioxide gas is also used together as part of the raw materials to obtain the urea synthesis solution.

6. The apparatus according to claim 4, further comprising:
a separation and purification facility for separating a separation gas containing carbon dioxide and ammonia from the urea synthesis solution obtained in the urea synthesis facility to obtain a purified aqueous urea solution after the separation; and
a concentration facility for evaporating water and minute remnants of ammonia and carbon dioxide from the purified aqueous urea solution to obtain a concentrated urea solution, IQ recover the evaporated water, ammonia and carbon dioxide and urea entrained in the evaporated gas as an aqueous solution, and Q treat excess water to obtain clean treated water,
wherein in the high-pressure absorption facility, part of the aqueous solution separated in the concentration facility is allowed to act as an absorbing solvent on the separation gas separated in the separation and purification facility, such that the obtained carbamate solution is used as part of the raw materials for urea synthesis.

7. A method for improving an existing apparatus for producing urea, the method comprising
adding to the existing apparatus, at least
a carbon dioxide separation facility to perform a carbon dioxide separation step of bringing a carbon dioxide-containing gas into contact with a lean solution, which is an absorbing solution containing low-concentration carbon dioxide, ammonia and water, to absorb and separate carbon dioxide contained in the gas into the lean solution to obtain a rich solution, which is an absorbing solution containing high-concentration carbon dioxide, ammonia and water, and
a rich solution stripping facility to perform a rich solution stripping step of stripping, at a temperature of 100 to 230° C. and a pressure of 1 to 16 MPaG, at least part of the rich solution obtained in the carbon dioxide separation step to gasify carbon dioxide to separate a gas containing high-concentration carbon dioxide, and to turn the rich solution into an aqueous solution containing low-concentration carbon dioxide and ammonia
to enable a method for producing urea, which further includes:
a high-pressure absorption step of bringing at least part of the gas separated in the rich solution stripping step into contact with ammonia at a temperature of 90 to 180° C. and a pressure of 1.0 to 10 MPaG to obtain a carbamate solution, and
a urea synthesis step of obtaining, at a temperature of 170 to 200° C. and a pressure of 13 to 25 MPaG, a urea synthesis solution using the carbamate solution obtained in the high-pressure absorption step as part of raw materials.

8. The method according to claim 7, comprising
further adding a regeneration facility to perform a regeneration step of separating a high-purity carbon dioxide gas from at least part of the rich solution obtained in the carbon dioxide separation facility other than the part of the rich solution to be stripped in the rich solution stripping facility,
wherein in the urea synthesis step, the high-purity carbon dioxide gas is also used together as part of the raw materials to obtain the urea synthesis solution.

9. The method according to claim 1, wherein
the rich solution stripping step is performed at a temperature of 130 to 210° C. and a pressure of 1 to 2.5 MPaG, and
the high-pressure absorption step is performed at a temperature of 90 to 120° C. and a pressure of 1.4 to 2.0 MPaG.

10. The apparatus according to claim 4, wherein
the stripping is performed in the rich solution stripping facility at a temperature of 130 to 210° C. and a pressure of 1 to 2.5 MPaG, and
the carbamate solution is obtained in the high-pressure absorption facility at a temperature of 90 to 120° C. and a pressure of 1.4 to 2.0 MPaG.

11. The method according to claim 7, wherein
the rich solution stripping step is performed at a temperature of 130 to 210° C. and a pressure of 1 to 2.5 MPaG, and
the high-pressure absorption step is performed at a temperature of 90 to 120° C. and a pressure of 1.4 to 2.0 MPaG.
